SEQUENCE LISTING (8 sequences)

SEQ ID NO: 1 - DNA, 20 bp, Artificial sequence
  Synthetic construct (primer)
acgagtaacgggacaaatgc

SEQ ID NO: 2 - DNA, 20 bp, Artificial sequence
  Synthetic construct (primer)
tccctaatctatccgcctga

SEQ ID NO: 3 - DNA, 23 bp, Artificial Sequence
  Synthetic construct
cgaatgtaacagacacggtctca

SEQ ID NO: 4 - DNA, 23 bp, Artificial Sequence
  Synthetic construct
cgaatgtaacagacacggtctca

SEQ ID NO: 5 - DNA, 23 bp, Artificial Sequence
  Synthetic construct
cgaatgtaacagacacggtctca

SEQ ID NO: 6 - DNA, 23 bp, Artificial Sequence
  Synthetic construct
tgagaccgtgtctgttacattcg

SEQ ID NO: 7 - protein, 224 aa, Pyrococcus furiosus
MKIGGIDEAGRGPAIGPLVVATVVVDEKNIEKLRNIGVKDSKQLTPHERKNLFSQITSIADDYKIVIVSPEEIDNRSGTMNELEVEKFALALNSLQIKPALIYADAADVDANRFASLIERRLNYKAKIIAEHKADAKYPVVSAASILAKVVRDEEIEKLKKQYGDFGSGYPSDPKTKKWLEEYYKKHNSFPPIVRRTWETVRKIEESIKAKKSQLTLDKFFKKP

SEQ ID NO: 8 - DNA, 23 bp, Artificial Sequence
  Synthetic construct
cgaatgtaacagacacggtctca

(19) United States
(12) Patent Application Publication
(10) Pub. No.: US 2012/0052495 A1
(43) Pub. Date: Mar. 1, 2012

(54) OLIGONUCLEOTIDES FOR DETECTING LISTERIA MONOCYTOGENES AND USE THEREOF

(75) Inventor: Jun LI, Baltimore, MD (US)

(73) Assignee: SAMSUNG TECHWIN CO., LTD., Changwon-city (KR)

(21) Appl. No.: 13/111,272

(22) Filed: May 19, 2011

Related U.S. Application Data
(60) Provisional application No. 61/378,117, filed on Aug. 30, 2010.

(51) Int. Cl. C12Q 1/68

(52) U.S. Cl. 435/6.11; 435/6.12

(57) ABSTRACT

Oligonucleotides suitable for detecting Listeria monocytogenes, and a kit and a method of efficiently detecting Listeria monocytogenes in a sample by using the oligonucleotides are provided.

### CROSS-REFERENCE TO RELATED APPLICATIONS

This application claims benefits from U.S. Provisional Patent Application No. 61/378,117, filed on Aug. 30, 2010, the content of which is hereby incorporated by reference in its entirety.

### FIELD

An oligonucleotide set and a kit for detecting Listeria monocytogenes and a method of detecting Listeria monocytogenes in a sample by using the same are disclosed.

### RELATED ART

Listeria monocytogenes bacteria are gram-positive, non-spore forming and motile bacilli and can grow in a wide temperature range of about −4° C. to about 45° C. and a wide pH range of about <5.5 to about 9.5. The Listeria genus contains six species, including Listeria monocytogenes, L. innocua, L. welshimeri, L. seeligeri, L. ivanovii, and L. grayi. Among these species of Listeria, L. monocytogenes is the cause of most human listeriosis cases. The immunocompromised, pregnant women, elderly, and neonates are susceptible to infection caused by this species. Typical symptoms of listeriosis include septicemia, meningitis and miscarriage.

Consumption of contaminated foods is the major cause of Listeria infection. There have been epidemics of various Listeria-induced infections caused by the consumption of contaminated foods, such as unpasteurized milk, contaminated cheese, coleslaw, and the like. Therefore, there is an increasing demand for a method of rapid, sensitive, and accurate detection of Listeria, specifically, Listeria monocytogenes in a sample, such as in a food, a surface wipe, or medical sample.

### SUMMARY

A composition, which is suitable for a rapid, sensitive and accurate detection of Listeria monocytogenes is disclosed. The composition includes a first oligonucleotide of the sequence of SEQ ID NO: 1, and a second oligonucleotide of the sequence of SEQ ID NO: 2.

According an embodiment, the composition may further contain a probe oligonucleotide of SEQ ID NO: 6 (TGAGACCGTGTCTrGTTACATTCG, wherein the nucleotide “rG” at position 14 is a ribonucleotide) or SEQ ID NO: 8 (CGAATGTAACAGACACGGTCTCA, wherein at least one of the ribonucleotides at positions 9, 10, 11, 12, and 13 is a ribonucleotide). In one embodiment, the probe oligonucleotide has a DNA sequence and an RNA sequence, and is one or more selected from the group consisting of oligonucleotides of SEQ ID NOs: 3-6: CGAATGTAArCAGACACGGTCTCA (SEQ ID NO: 3), wherein “rC” at position 10 is a ribonucleotide, CGAATGTArArCrArGACACGGTCTCA (SEQ ID NO: 4), wherein “rA,” “rC,” “rA,” and “rG” at positions 9, 10, 11, and 12, respectively, are ribonucleotides, CGAATGTAArCrArGrACACGGTC-TCA (SEQ ID NO: 5), wherein “rC,” “rA,” “rG,” and “rA” at positions 10, 11, 12, and 13, respectivelyare ribonucleotides, and TGAGACCGTGTCTrGTTACATTCG (SEQ ID NO: 6), wherein the nucleotide “rG” at position 14 is a ribonucleotide.

In still another embodiment, a kit for detecting Listeria monocytogenes in a sample, the kit containing the above composition is provided. The kit may further include an amplifying activity and an RNase H. In an embodiment, the kit may further comprise a reverse transcriptase activity for reverse transcription of a target Listeria monocytogenes RNA sequence.

In another embodiment, a method of detecting Listeria monocytogenes in a sample is provided. The method includes (a) amplifying a target nucleic acid of Listeria monocytogenes in the sample to produce an increased number of copies of the target nucleic acid, the amplification including hybridizing a first primer of SEQ ID NO: 1 and a second primer of SEQ ID NO: 2 to the target nucleic acid in the sample to obtain a hybridized product of the target nucleic acid and the primers, and extending the first and the second primers of the hybridized product using a template-dependent nucleic acid polymerase to produce an extended primer product; (b) hybridizing the target nucleic acid to at least one probe oligonucleotide which is capable of being hybridized to the target nucleic acid to obtain a hybridized product of the target nucleic acid:probe oligonucleotide, said probe comprising a DNA sequence and an RNA sequence, and being coupled to a detectable marker; (c) contacting the hybridized product of the target nucleic acid:probe with an RNase H to cleave the probe, resulting in probe fragment dissociation from the target nucleic acid; and (d) detecting the detectable marker. The probe oligonucleotide may be the oligonucleotide of SEQ ID NOs: 6 or 8. The probe oligonucleotide may be one of oligonucleotides of SEQ ID NOs: 3-6. The probe oligonucleotide may be labeled with a detectable marker, for example a fluorescence resonance energy transfer pair.

In another embodiment, a method of detecting a target RNA sequence of Listeria monocytogenes in a sample is provided. The method includes (a) reverse transcribing the Listeria monocytogenes target RNA in the presence of a reverse transcriptase activity and the reverse amplification primer to produce a target cDNA of the target RNA; (b) amplifying the target cDNA sequence to produce an increased number of copies of the target nucleic acid, the amplification including hybridizing a first primer of SEQ ID NO: 1 and a second primer of SEQ ID NO: 2 to the target cDNA to obtain a hybridized product of the target nucleic acid and the primers, and extending the first and the second primers of the hybridized product using a template-dependent nucleic acid polymerase to produce an extended primer product; (c) hybridizing the target nucleic acid to at least one probe oligonucleotide which is substantially complimentary to the target cDNA to obtain a hybridized product of the target nucleic acid:probe oligonucleotide, wherein the probe comprises a DNA sequence and an RNA sequence and is coupled to a detectable marker; (d) contacting the hybridized product of the target nucleic acid:probe oligonucleotide with an RNase H to cleave the probe; and (e) detecting an increase in the emission of a signal from the detectable marker on the probe, wherein the increase in signal indicates the presence of the Listeria monocytogenes target RNA in the sample.

Amplification of a target sequence in a sample may be performed by using any nucleic acid amplification method, such as the Polymerase Chain Reaction (U.S. Pat. Nos. 4,683,195, 4,683,202, and 4,800,159) or by using amplification reactions such as Ligase Chain Reaction (Proc. Natl. Acad. Sci. USA 88:189-193), Self-Sustained Sequence Replication (Guatelli et al., 1990, Proc. Natl. Acad. Sci. USA 87:1874-1878), Strand Displacement Amplification (U.S. Pat. Nos. 5,270,184, en 5,455,166), Transcriptional Amplification System (Kwoh et al., Proc. Natl. Acad. Sci. USA 86:1173-1177), Q-Beta Replicase (Lizardi et al., 1988, Bio/Technology 6:1197), Nucleic Acid Sequence Based Amplification (NASBA), Cleavage Fragment Length Polymorphism (U.S. Pat. No. 5,719,028), Isothermal and Chimeric Primer-initiated Amplification of Nucleic Acid (U.S. Pat. No. 6,951,722), Ramification-extension Amplification Method (U.S. Pat. Nos. 5,719,028 and 5,942,391) or other suitable methods for amplification of nucleic acid.

The amplification, hybridization, and contacting steps may be performed simultaneously or sequentially.

In an embodiment, the sample containing Listeria monocytogenes may be cultured in an enrichment medium before the amplification, to enhance growth of the Listeria monocytogenes. Such enrichment medium may contain, per 1 L of distilled water, about 10 to about 40 g of tryptic soy broth, about 1 to about 10 g of yeast extract, and about 1 to about 10 g of lithium chloride. The enrichment medium may further contain at least one component selected from the group consisting of about 1 to about 10 g of beef extract, and/or a vitamin mix containing about 0.01 to about 0.5 mg of riboflavin, about 0.5 to about 1.5 mg of thiamine and about 0.01 to about 1.5 mg of biotin; about 1 to about 5 g of pyruvate; and about 0.01 to about 1 g of ferric ammonium citrate. The enrichment medium may further comprise a buffer compound, for example 3-(N-morpholino)propanesulfonic acid (MOPS) and a sodium salt thereof.

In another embodiment, the enrichment medium may contain about 1 to about 10 mg of acriflavine, about 5 to about 15 mg of polymyxin B, and about 10 to about 30 mg of ceftazidime, per 1 L of distilled water. For example, the enrichment medium may contain, per 1 L of distilled water, about 10 to about 40 g of tryptic soy broth, about 1 to about 10 g of yeast extract, about 1 to about 10 g of lithium chloride; about 1 to about 10 g of beef extract and/or a vitamin mix containing about 0.01 to about 0.5 mg of riboflavin, about 0.5 to about 1.5 mg of thiamine, and about 0.01 to about 1.5 mg of biotin; about 1 to about 5 g of pyruvate or a salt thereof; about 0.1 to about 1 g of ferric ammonium citrate; about 4 g of 3-(N-morpholino)propanesulfonic acid (MOPS) and about 7.1 g of sodium MOPS; and about 1 to about 10 mg of acriflavine, about 5 to about 15 mg of polymyxin B, and about 10 to about 30 mg of ceftazidime. In an embodiment, the enrichment medium does not contain one of esculin or peptone, or both.

In still another embodiment, the enrichment medium may contain, per 1 L of distilled water, about 30 g of tryptic soy broth, about 6 g of yeast extract, about 1 to about 10 g of lithium chloride; about 5 g of beef extract and/or a vitamin mix containing about 0.1 mg of riboflavin, about 1.0 mg of thiamine, and about 1.0 mg of biotin; about 2 g of sodium pyruvate; about 0.2 g of ferric ammonium citrate; about 4 g of 3-(N-morpholino)propanesulfonic acid (MOPS) and about 7.1 g of sodium MOPS; and about 5 mg of acriflavine, about 10 mg of polymyxin B, and about 20 mg of ceftazidime.

In another embodiment, the enrichment medium may be brain-heart infusion broth or tryptic soy broth containing 0.6% yeast extract.

The sample may be a food sample, a medical sample, or a surface wipe.

### BRIEF DESCRIPTION OF THE DRAWINGS

FIG. 1 is a graph illustrating the real-time polymerase chain reaction (PCR) results on 61 L. monocytenges strains;

FIG. 2 is a graph illustrating the real-time PCR results on 56 other organisms;

FIGS. 3(A) and FIG. 3(B) show the results of sensitivity tests using 1 gene copy, wherein FIG. 4(A) shows fluorescence and FIG. 4(B) shows Cp value; and

FIGS. 4(A) and FIG. 4(B) show the results of sensitivity tests using 1×10⁴ cfu/mL of L. monocytogenes, wherein FIG. 4(A) shows fluorescence and FIG. 4(B) shows Cp value.

### DETAILED DESCRIPTION

The practice of the embodiments described herein employs, unless otherwise indicated, conventional molecular biological techniques within the skill of the art. Such techniques are well known to the skilled worker, and are explained fully in the literature. See, e.g., Ausubel, et al., ed., Current Protocols in Molecular Biology, John Wiley & Sons, Inc., N.Y. (1987-2008), including all supplements; Sambrook, et al., Molecular Cloning: A Laboratory Manual, 2nd Edition, Cold Spring Harbor, N.Y. (1989).

Unless defined otherwise, all technical and scientific terms used herein have the same meaning as is commonly understood by one of skill in the art. The specification also provides definitions of terms to help interpret the disclosure and claims of this application. In the event a definition is not consistent with definitions elsewhere, the definition set forth in this application will control.

The term “amplification” used herein refers to any process for increasing the number of copies of nucleotide sequences. Nucleic acid amplification describes a process whereby nucleotides are incorporated into nucleic acids, for example, DNA or RNA.

The term “nucleotide” used herein refers to a base-sugar-phosphate combination. Nucleotides are the monomeric units of nucleic acids, for example, DNA or RNA. The term “nucleotide” includes ribonucleoside triphosphates, such as rATP, rCTP, rGTP, or rUTP, and deoxy-ribonucleotide triphosphates, such as dATP, dCTP, dGTP, or dTTP.

The term “nucleoside” used herein refers to a base-sugar combination, i.e., a nucleotide lacking phosphate moieties. The terms “nucleoside” and “nucleotide” are used interchangeably in the field. For example, the nucleotide deoxyuridine, dUTP, is a deoxynucleoside triphosphate. It serves as a DNA monomer, for example, being dUMP or deoxyuridine monophosphate, after being inserted into DNA. In this regard, even though no dUTP moiety is present in the result DNA, dUTP may be considered as having been inserted.

The term “polymerase chain reaction (PCR)” generally refers to an amplification method for increasing the number of copies of target nucleic acid(s) in a sample. The procedure is described in detail in U.S. Pat. Nos. 4,683,202, 4,683,195, 4,800,159, and 4,965,188, the contents of which are incorporated herein in their entirety. The sample may include a single nucleic acid or multiple nucleic acids. In general, PCR involves incorporating at least two extendible primer nucleic acids into a reaction mixture containing target nucleic acid(s). The primers are complementary to opposite strands of a double-stranded target sequence. The reaction mixture is subjected to thermal cycling in the presence of a nucleic acid polymerase and nucleic acid monomers, for example, in the presence of dNTP's and/or rNTP's, to amplify the target nucleic acid by extension of the primers. In general, the thermal cycling may involve: annealing to hybridize the primer and target nucleic acid; extending the primers using a nucleic acid polymerase; and denaturating the hybridized primer extension product and the target nucleic acid. The term “reverse transcriptase-PCR(RT-PCR)” is a PCR that uses an RNA template and a reverse transcriptase, or an enzyme having reverse transcriptase activity, to first generate a single stranded cDNA molecule prior to the multiple cycles of DNA-dependent DNA polymerase primer extension. The term “multiplex PCR” refers to PCRs that produce more than two amplified target products in a single reaction, typically by the inclusion of more than two primers.

The term “nucleic acid” used herein refers to a polymer including more than two nucleotides. The term “nucleic acid” is used interchangeably with “polynucleotide” or “oligonucleotide”. Nucleic acids include DNA and RNA. The structure of nucleic acids may be double-stranded and/or single-stranded.

The term “nucleic acid analog” used herein refers to a nucleic acid that contains at least one nucleotide analog and/or at least one phosphate ester analog and/or at least one pentose sugar analog. Examples of nucleic acid analogues include nucleic acids in which the phosphate ester and/or sugar phosphate ester linkages are replaced with other types of linkages, such as N-(2-aminoethyl)-glycine amides and other amides. Nucleic acid analogs refer to a nucleic acid that contains at least one nucleotide analog and/or at least one phosphate ester analog and/or at least one pentose sugar analog and may form a double helix by hybridization.

The terms “annealing” and “hybridization” used herein are interchangeable and refer to the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

The term “probe” used herein refers to a nucleic acid having a sequence complementary to a target nucleic acid sequence and capable of hybridizing to the target nucleic acid to form a duplex. The sequence of the probe may be fully or completely complementary to the target nucleic acid sequence. The probe may be labeled so that the target nucleic acid may be detected simultaneously with PCR.

The terms “target nucleic acid” or “target sequence” used herein includes a full length or a fragment of a target nucleic acid that may be amplified and/or detected. A target nucleic acid may be present between two primers that are used for amplification.

The term “hybrid oligonucleotide” used herein with regard to an oligonucleotide means an oligonucleotide molecule which contains a DNA and an RNA portion within a single molecule. The hybrid oligonucleotide may contain more than one DNA portion and one RNA portion, for example a DNA-RNA, RNA-DNA, or DNA-RNA-DNA oligonucleotide.

In embodiments, an oligonucleotide set for detecting Listeria monocytogenes includes a primer of SEQ ID NO:1; a primer of SEQ ID NO:2; and at least one probe selected from the group consisting of oligonucleotides of SEQ ID NOs: 3-6.

The primer pair of SEQ ID NOs: 1 and 2 have sequences complementary to the respective opposite strands of a target nucleic acid, and may define a target nucleic acid of about 184 in length. The primer pair is complementary to the internalin A (inlA) gene of L. monocytogenes and thus is available to specifically amplify the target nucleic acid in the inlA gene. InlA belongs to a large group of a surface-localized leucine-rich repeat (LRR) identified in the Listeria genome. InlA allows L. monocytogenes to enter into and adsorb non-phagocytic cells such as endothelial cells of human internal organs so as to induce intake in the endothelial cells. Listeria monocytogenes genomic DNA can be prepared by the method described by Flamm et al., and the InlA gene can be cloned from genomic DNA by PCR.

The InlA gene is of 2403 bp and codes a peptide of 801 amino acid residues. When used for amplification, the primer pair can amplify target nucleic acid sequences of any L. monocytogenes, but not the target nucleic acid sequences of other Listeria monocytogenes and non-Listeria monocytogenes When used for amplification, the primer pair can amplify target nucleic acid sequences of any Listeria species of the Listeria genus, but not the target nucleic acid sequences of non-Listeria monocytogenes Thus, the primer pair specifically amplifies target nucleic acids of Listeria monocytogenes with single copy sensitivity.

In one embodiment, the probe may have a DNA-RNA-DNA hybrid structure. The probe may be a nucleic acid or a nucleic acid analog. The probe also may be a protected nucleic acid. For example, a DNA or RNA portion of the probe may be partially methylated to be resistant to degradation by an RNA-specific enzyme, for example, an RNase H.

The probe may be modified. For example, the base portion of the probe may be partially or fully methylated. Such modifications may inhibit enzymatic or chemical degradation. The 5′ end or 3′ end —OH group of the nucleic acid probe may be blocked. The 3′ end OH group of the nucleic acid probe may be blocked, thus being rendered incapable of extension by a template-dependant nucleic acid polymerase.

The probe may have a detectable label. The detectable label may be any chemical moiety detectable by any method known in the field. Examples of detectable labels include any moiety detectable by spectroscopy, photochemistry, or by biochemical, immunochemical or chemical means. A suitable method of labeling the nucleic acid probe may be selected according to the type of the label and the positions of the label and probe. Examples of labels include enzymes, enzyme substrates, radioactive substance, fluorescent dyes, chromophores, chemiluminescent labels, electrochemical luminescent label, ligands having specific binding partners, and other labels that interact with each other to increase, vary or reduce the intensity of a detection signal. These labels are durable throughout the thermal cycling for PCR.

The detectable label may be a fluorescence resonance energy transfer (FRET) pair. The detectable label is a FRET pair including a fluorescent donor and a fluorescent acceptor separated by an appropriate distance, and in which donor fluorescence emission is quenched by the acceptor. However, when the donor-acceptor pair is dissociated by cleavage, donor fluorescence emission is enhanced. A donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Examples of donor chromophores include FAM, TAMRA, VIC, JOE, Cy3, Cy5, and Texas Red. Acceptor chromophores are chosen so that their excitation spectra overlap with the emission spectrum of the donor. An example of such a pair is FAM-TAMRA. In addition, an example of the detectable label is a non-fluorescent acceptor that will quench a wide range of donors. Other examples of appropriate donor-acceptor FRET pairs will be known to those of skill in the art.

In an embodiment, the oligonucleotide probe may be present as a soluble form or free form in a solution. In another embodiment, the oligonucleotide probe can be attached to a solid support. Different probes may be attached to the solid support and may be used to simultaneously detect different target sequences in a sample. Reporter molecules having different fluorescence wavelengths can be used on the different probes, thus enabling hybridization to the different probes to be separately detected.

Examples of preferred types of solid supports for immobilization of the oligonucleotide probe include polystyrene, avidin coated polystyrene beads cellulose, nylon, acrylamide gel and activated dextran, controlled pore glass (CPG), glass plates and highly cross-linked polystyrene. These solid supports are preferred for hybridization and diagnostic studies because of their chemical stability, ease of functionalization and well defined surface area. Solid supports such as controlled pore glass (500 Å, 1000 Å) and non-swelling high cross-linked polystyrene (1000 Å) are particularly preferred in view of their compatibility with oligonucleotide synthesis.

The oligonucleotide probe may be attached to the solid support in a variety of manners. For example, the probe may be attached to the solid support by attachment of the 3′ or 5′ terminal nucleotide of the probe to the solid support. However, the probe may be attached to the solid support by a linker which serves to separate the probe from the solid support. The linker is most preferably at least 30 atoms in length, more preferably at least 50 atoms in length.

Hybridization of a probe immobilized to a solid support generally requires that the probe be separated from the solid support by at least 30 atoms, more-preferably at least 50 atoms. In order to achieve this separation, the linker generally includes a spacer positioned between the linker and the 3′ nucleoside. For oligonucleotide synthesis, the linker arm is usually attached to the 3′-OH of the 3′ nucleoside by an ester linkage which can be cleaved with basic reagents to free the oligonucleotide from the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. Alternatively, polymers such as functionalized polyethylene glycol can be used as the linker. Such polymers are preferred over homopolymeric oligonucleotides because they do not significantly interfere with the hybridization of probe to the target oligonucleotide. Polyethylene glycol is particularly preferred because it is commercially available, soluble in both organic and aqueous media, easy to functionalize, and is completely stable under oligonucleotide synthesis and post-synthesis conditions.

The linkages between the solid support, the linker and the probe are preferably not cleaved during removal of base protecting groups under basic conditions at high temperature. Examples of preferred linkages include carbamate and amide linkages. Immobilization of a probe is well known in the art and one skilled in the art may determine the immobilization conditions.

According to one embodiment of the method, the hybridization probe is immobilized on a solid support. The oligonucleotide probe is contacted with a sample of nucleic acids under conditions favorable for hybridization. In an unhybridized state, the fluorescent label is quenched by the acceptor. Upon hybridization to the target, the fluorescent label is separated from the quencher and the fluorescence emission is enhanced.

Immobilization of the hybridization probe to the solid support also enables the target sequence hybridized to the probe to be readily isolated from the sample. In later steps, the isolated target sequence may be separated from the solid support and processed (e.g., purified, amplified) according to methods well known in the art depending on the particular needs of the researcher.

In an embodiment, the oligonucleoride set suitable for detecting Listeria monocytogenes may include a primer of SEQ ID NO. 1; a primer of SEQ ID NO. 2; and a probe of SEQ ID NO. 3.

The oligonucleotide set may be used for amplification and detection of target nucleic acids. The amplification may include extending the primers using a template-dependent polymerase, which results in the formation of PCR fragment or amplicon. The amplification can be accomplished by any method selected from the group consisting of Polymerase Chain Reaction or by using amplification reactions such as Ligase Chain Reaction, Self-Sustained Sequence Replication, Strand Displacement Amplification, Transcriptional Amplification System, Q-Beta Replicase, Nucleic Acid Sequence Based Amplification (NASBA), Cleavage Fragment Length Polymorphism, Isothermal and Chimeric Primer-initiated Amplification of Nucleic Acid, Ramification-extension Amplification Method or other suitable methods for amplification of nucleic acid. The amplification may include simultaneous real-time detection of target nucleic acids

The term “PCR fragment” or “amplicon” refers to a polynucleotide molecule (or collectively the plurality of molecules) produced following the amplification of a particular target nucleic acid. A PCR fragment is typically, but not exclusively, a DNA PCR fragment. A PCR fragment can be single-stranded or double-stranded, or a mixture thereof in any concentration ratio. A PCR fragment can be 100-500 nucleotides or more in length.

An amplification “buffer” is a compound added to an amplification reaction which modifies the stability and/or activity of one or more components of the amplification reaction by regulating the amplification reaction. The buffering agents of the invention are compatible with PCR amplification and RNase H cleavage activity. Examples of buffers include, but are not limited to, HEPES ((4-(2-hydroxyethyl)-1-piperazineethanesulfonic acid), MOPS (3-(N-morpholino)-propanesulfonic acid), and acetate or phosphate containing buffers and the like. In addition, PCR buffers may generally contain up to about 70 mM KCl and about 1.5 mM or higher MgCl₂, and about 50-200 μM each of dATP, dCTP, dGTP and dTTP. The buffers of the invention may contain additives to optimize efficient reverse transcriptase-PCR or PCR reactions.

An additive is a compound added to a composition which modifies the stability and/or activity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, NaI, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll, aurintricarboxylic acid, Tween 20, Tween 21, Tween 40, Tween 60, Tween 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction. Additives may be optionally added to improve selectivity of primer annealing provided the additives do not interfere with the activity of RNase H.

As used herein, the term “thermostable,” as applied to an enzyme, refers to an enzyme that retains its biological activity at elevated temperatures (e.g., at 55° C. or higher), or retains its biological activity following repeated cycles of heating and cooling. Thermostable polynucleotide polymerases find particular use in PCR amplification reactions.

As used herein, a “thermostable polymerase” is an enzyme that is relatively stable to heat and eliminates the need to add enzyme prior to each PCR cycle. Non-limiting examples of thermostable polymerases may include polymerases isolated from the thermophilic bacteria Thermus aquaticus (Taq polymerase), Thermus thermophilus (Tth polymerase), Thermococcus litoralis (Tli or VENT polymerase), Pyrococcus furiosus (Pfu or DEEPVENT polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME polymerase) Thermotoga neapolitana (Tne polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq polymerase. Many variants of Taq polymerase with enhanced properties are known and include AmpliTaq, AmpliTaq Stoffel fragment, SuperTaq, SuperTaq plus, LA Taq, LApro Taq, and EX Taq.

One of the most widely used techniques to study gene expression exploits first-strand cDNA for mRNA sequence(s) as template for amplification by the PCR. This method, often referred to as reverse transcriptase-PCR, exploits the high sensitivity and specificity of the PCR process and is widely used for detection and quantification of RNA.

The reverse transcriptase-PCR procedure, carried out as either an end-point or real-time assay, involves two separate molecular syntheses: (i) the synthesis of cDNA from an RNA template; and (ii) the replication of the newly synthesized cDNA through PCR amplification. To attempt to address the technical problems often associated with reverse transcriptase-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcriptase-PCR procedure (e.g., two step reverse transcriptase-PCR), reverse transcription is performed as an independent step using the optimal buffer condition for reverse transcriptase activity. Following cDNA synthesis, the reaction is diluted to decrease MgCl₂, and deoxyribonucleoside triphosphate (dNTP) concentrations to conditions optimal for Taq DNA Polymerase activity, and PCR is carried out according to standard conditions (see U.S. Pat. Nos. 4,683,195 and 4,683,202). By contrast, “coupled” reverse transcriptase PCR methods use a common buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn²⁺ then PCR is carried out in the presence of Mg²⁺ after the removal of Mn²⁺ by a chelating agent. Finally, the “continuous” method (e.g., one step reverse transcriptase-PCR) integrates the three reverse transcriptase-PCR steps into a single continuous reaction that avoids the opening of the reaction tube for component or enzyme addition. Continuous reverse transcriptase-PCR has been described as a single enzyme system using the reverse transcriptase activity of thermostable Taq DNA Polymerase and Tth polymerase and as a two enzyme system using AMV reverse transcriptase and Taq DNA Polymerase wherein the initial 65° C. RNA denaturation step was omitted.

The first step in real-time, reverse-transcription PCR is to generate the complementary DNA strand using one of the template specific DNA primers. In traditional PCR reactions this product is denatured, the second template specific primer binds to the cDNA, and is extended to form duplex DNA. This product is amplified in subsequent rounds of temperature cycling. To maintain the highest sensitivity it is important that the RNA not be degraded prior to synthesis of cDNA. The presence of RNase H in the reaction buffer will cause unwanted degradation of the RNA:DNA hybrid formed in the first step of the process because it can serve as a substrate for the enzyme. There are two major methods to combat this issue. One is to physically separate the RNase H from the rest of the reverse-transcription reaction using a barrier such as wax that will melt during the initial high temperature DNA denaturation step. A second method is to modify the RNase H such that it is inactive at the reverse-transcription temperature, typically 45-55° C. Several methods are known in the art, including reaction of RNase H with an antibody, or reversible chemical modification. For example, a hot start RNase H activity as used herein can be an RNase H with a reversible chemical modification produced after reaction of the RNase H with cis-aconitic anhydride under alkaline conditions. When the modified enzyme is used in a reaction with a Tris based buffer and the temperature is raised to 95° C. the pH of the solution drops and RNase H activity is restored. This method allows for the inclusion of RNase H in the reaction mixture prior to the initiation of reverse transcription.

Additional examples of RNase H enzymes and hot start RNase H enzymes that can be employed in the invention are described in U.S. Patent Application No. 2009/0325169 to Walder et al., the content of which is incorporated herein in its entirety.

One step reverse transcriptase-PCR provides several advantages over uncoupled reverse transcriptase-PCR. One step reverse transcriptase-PCR requires less handling of the reaction mixture reagents and nucleic acid products than uncoupled reverse transcriptase-PCR (e.g., opening of the reaction tube for component or enzyme addition in between the two reaction steps), and is therefore less labor intensive, reducing the required number of person hours. One step reverse transcriptase-PCR also reduces the risk of contamination. The sensitivity and specificity of one-step reverse transcriptase-PCR has proven well suited for studying expression levels of one to several genes in a given sample or the detection of pathogen RNA. Typically, this procedure has been limited to use of gene-specific primers to initiate cDNA synthesis.

The ability to measure the kinetics of a PCR reaction by real-time detection in combination with these reverse transcriptase-PCR techniques has enabled accurate and precise determination of RNA copy number with high sensitivity. This has become possible by detecting the reverse transcriptase-PCR product through fluorescence monitoring and measurement of PCR product during the amplification process by fluorescent dual-labeled hybridization probe technologies, such as the 5′ fluorogenic nuclease assay (“Taq-Man”) or endonuclease assay (sometimes referred to as, “CataCleave”), discussed below.

Post-amplification amplicon detection is both laborious and time consuming. Real-time methods have been developed to monitor amplification during the PCR process. These methods typically employ fluorescently labeled probes that bind to the newly synthesized DNA or dyes whose fluorescence emission is increased when intercalated into double stranded DNA.

The probes are generally designed so that donor emission is quenched in the absence of target by fluorescence resonance energy transfer (FRET) between two chromophores. The donor chromophore, in its excited state, may transfer energy to an acceptor chromophore when the pair is in close proximity. This transfer is always non-radiative and occurs through dipole-dipole coupling. Any process that sufficiently increases the distance between the chromophores will decrease FRET efficiency such that the donor chromophore emission can be detected radiatively. Common donor chromophores include FAM, TAMRA, VIC, JOE, Cy3, Cy5, and Texas Red. Acceptor chromophores are chosen so that their excitation spectra overlap with the emission spectrum of the donor. An example of such a pair is FAM-TAMRA. There are also non fluorescent acceptors that will quench a wide range of donors. Other examples of appropriate donor-acceptor FRET pairs will be known to those skilled in the art.

Common examples of FRET probes that can be used for real-time detection of PCR include molecular beacons (e.g., U.S. Pat. No. 5,025,517), TaqMan probes (e.g., U.S. Pat. Nos. 5,210,015 and 5,487,972), and CataCleave probes (e.g., U.S. Pat. No. 5,763,181). The molecular beacon is a single stranded oligonucleotide designed so that in the unbound state the probe forms a secondary structure where the donor and acceptor chromophores are in close proximity and donor emission is reduced. At the proper reaction temperature the beacon unfolds and specifically binds to the amplicon. Once unfolded, the distance between the donor and acceptor chromophores increases such that FRET is reversed and donor emission can be monitored using specialized instrumentation. TaqMan and CataCleave technologies differ from the molecular beacon in that the FRET probes employed are cleaved such that the donor and acceptor chromophores become sufficiently separated to reverse FRET.

TaqMan technology employs a single stranded oligonucleotide probe that is labeled at the 5′ end with a donor chromophore and at the 3′ end with an acceptor chromophore. The DNA polymerase used for amplification must contain a 5′->3′ exonuclease activity. The TaqMan probe binds to one strand of the amplicon at the same time that the primer binds. As the DNA polymerase extends the primer the polymerase will eventually encounter the bound TaqMan probe. At this time the exonuclease activity of the polymerase will sequentially degrade the TaqMan probe starting at the 5′ end. As the probe is digested the mononucleotides comprising the probe are released into the reaction buffer. The donor diffuses away from the acceptor and FRET is reversed. Emission from the donor is monitored to identify probe cleavage. Because of the way TaqMan works a specific amplicon can be detected only once for every cycle of PCR. Extension of the primer through the TaqMan target site generates a double stranded product that prevents further binding of TaqMan probes until the amplicon is denatured in the next PCR cycle.

U.S. Pat. No. 5,763,181, the content of which is incorporated herein by reference, describes another real-time detection method (referred to as “CataCleave”). CataCleave technology differs from TaqMan in that cleavage of the probe is accomplished by a second enzyme that does not have polymerase activity. The CataCleave probe has a sequence within the molecule which is a target of an endonuclease, such as a restriction enzyme or RNase. In one example, the CataCleave probe has a chimeric structure where the 5′ and 3′ ends of the probe are constructed of DNA and the cleavage site contains RNA. The DNA sequence portions of the probe are labeled with a FRET pair either at the ends or internally. The PCR reaction includes an RNase H enzyme that will specifically cleave the RNA sequence portion of a RNA-DNA duplex. After cleavage, the two halves of the probe dissociate from the target amplicon at the reaction temperature and diffuse into the reaction buffer. As the donor and acceptors separate FRET is reversed in the same way as the TaqMan probe and donor emission can be monitored. Cleavage and dissociation regenerates a site for further CataCleave binding. In this way it is possible for a single amplicon to serve as a target or multiple rounds of probe cleavage until the primer is extended through the CataCleave probe binding site.

In embodiments, the probe used in the method is a CataCleave probe. Examples of suitable CataCleave probes include oligonucleotides comprising the sequence of one of SEQ ID NOS: 3-6.

In embodiments, a kit for detecting Listeria monocytogenes in a sample includes the oligonucleotides described above.

The kit may further include a reagent for nucleic acid amplification. The reagent may further include at least one selected from the group consisting of dNTP's, rNTP's, a nucleic acid polymerase, a uracil N-glycosylase (UNG) enzyme, a buffer, and a cofactor (for example, Mg²⁺). The nucleic acid polymerase may be selected from the group consisting of a DNA polymerase, a RNA polymerase, and a reverse transcriptase. The nucleic acid polymerase may be thermostable. The nucleic acid polymerase may retain its activity at elevated temperatures, for example, at 95° C. or higher. Thermostable DNA polymerases may be isolated from heat-resistant bacteria selected from the group consisting of Thermus aquaticus, Thermus flavus, Thermus ruber, Thermus thermophilus, Bacillus stearothermophilus, Thermus lacteus, Thermus rubens, Thermotoga maritima, Thermococcus littoralis, and Methanothermus fervidus. An example of a thermostable DNA polymerase is a Taq polymerase. The Taq polymerase is known to have optimal activity at about 70° C.

When the probe is hybridized to a target DNA, the Listeria monocytogenes detection kit may further include a factor specifically cleaving the RNA portion of the DNA-RNA hybrid. The cleaving factor may be RNase H. The cleaving factor may cleave specifically or nonspecifically the RNA portion. A specific RNA cleaving factor may be RNase HI. A nonspecific RNA cleaving factor may be RNase HII. RNase H may hydrolyze RNA in the RNA-DNA hybrid. For RNase H activity, a divalent ion (for example, Mg²⁺, Mn²⁺) is required. The RNase H cleaves RNA 3′-O—P linkages to produce 3′-hydroxyl and 5′-phosphate end products. The RNase H may be selected from the group consisting of a Pyrococcus furiosus RNase HII, a Pyrococcus horikoshi RNase HII, a Thermococcus litoralis RNase HI, and a Thermus thermophilus RNase HI. The Pyrococcus furiosus RNase HII may have an amino acid sequence of SEQ ID NO. 15. The RNase H may be thermostable. For example, the RNase H may retain its activity during a denaturation process in PCR. The cleaving factor may be a reversibly modified form of a thermostable RNase HII, which is inactive in its modified form and active in its unmodified form, wherein the modification is a coupling of the RNase HII to a ligand, crosslinking of the RNase HII, or chemical reaction of an amino acid residue in the RNase HII, and wherein the enzymatic activity of the modified RNase HII is restored by heating or adjusting pH of a sample containing the RNase HII.

When the RNA portion of the probe that contains a DNA sequence and an RNA sequence is cleaved by the cleaving factor, dissociation may occur. Such dissociation may naturally occur due to a decrease in the melting temperature of the cleaved complex or may be facilitated by a factor, such as temperature elevation. Dissociated fragments may be detected by any method known in the field.

In embodiments, a method of detecting Listeria monocytogenes in a sample includes: (a) amplifying a target nucleic acid of Listeria monocytogenes in the sample to produce an increased number of copies of the target nucleic acid, the amplifying including hybridizing a first primer of SEQ ID NO: 1 and a second primer of SEQ ID NO: 2 to the target nucleic acid in the sample to obtain a hybridized product of the target nucleic acid and the primers, and extending the first and the second primers of the hybridized product using a template-dependent nucleic acid polymerase to produce an extended primer product; (b) hybridizing the target nucleic acid to at least one probe oligonucleotide which is capable of being hybridized to the target nucleic acid to obtain a hybridized product of the target nucleic acid:probe oligonucleotide, wherein the probe contains an RNA sequence and a DNA sequence, and is coupled to a detectable marker; (c) contacting the hybridized product of the target nucleic acid:probe with RNase H to cleave the probes, resulting in probe fragment dissociation from the target nucleic acid; and (d) detecting the detectable marker.

Amplification of a target sequence in a sample may be performed by using any nucleic amplification method, such as the Polymerase Chain Reaction or by using amplification reactions such as Ligase Chain Reaction, Self-Sustained Sequence Replication, Strand Displacement Amplification, Transcriptional Amplification System, Q-Beta Replicase, Nucleic Acid Sequence Based Amplification (NASBA), Cleavage Fragment Length Polymorphism, Isothermal and Chimeric Primer-initiated Amplification of Nucleic Acid, Ramification-extension Amplification Method or other suitable methods for amplification of nucleic acid.

In an embodiment, the method includes amplifying a target nucleic acid fragment of Listeria monocytogenes, the amplifying including hybridizing a first primer of SEQ ID NO. 1 and a second primer of SEQ ID NO. 2 to the target nucleic acid in the sample to obtain a hybridized product; and extending the primers of the hybridized product using a template-dependent nucleic acid polymerase to produce an extended primer product; hybridizing the target nucleic acid fragment to a probe of SEQ ID NO: 6 or 8 to obtain a hybridized product; contacting the hybridized product from the target nucleic acid fragment and the probe to a RNase H to cleave the probes, resulting in a probe fragment dissociating from the hybridized product; and detecting the detectable marker.

Hereinafter, the method will now be described in greater detail. The method includes amplifying a target nucleic acid fragment of Listeria monocytogenes, the amplification including hybridizing a first primer of SEQ ID NO:1 and a second primer of SEQ ID NO:2 to the target nucleic acid in the sample to obtain a hybridized product; and extending the primers of the hybridized product depending on a template using a template-dependent nucleic acid polymerase to produced an extended primer product.

The hybridization may be conducted in a liquid medium. A suitable liquid medium may be selected according to the requirement(s). The liquid medium may be, for example, water, a buffer, or a PCR mixture. Nonlimiting examples of buffers include PBS, Tris, MOPS and Tricine. The hybridization may be conducted under the conditions to facilitate the binding of the primer and the target nucleic acid, for example, at low temperatures and low salt concentrations. Those conditions to facilitate hybridization are known in the field. The target nucleic acid may be a single-stranded or double-stranded nucleic acid. For example, a double-stranded target nucleic acid may be denaturated into separate single strands. The target nucleic acid may be DNA or RNA.

The extending of the primer depending on a template refers to polymerization, which is known in the field. The nucleic acid polymerase may be thermostable.

The method of detecting Listeria monocytogenes includes hybridizing the target nucleic acid fragment to at least one probe selected from the group consisting of oligonucleotides of SEQ ID NOS: 3-6 to obtain a hybridized product. The probes as described above may be used. The probe may be labeled with a detectable marker, for example, an optically detectable marker. Detectable markers are known in the art and may be suitably selected. For example, a FRET pair may be used for the purpose of detecting the target sequence in an embodiment of the invention.

The hybridization may be conducted in a liquid medium. A suitable liquid medium may be selected according to the requirement(s). The liquid medium may be, for example, water, a buffer, or a PCR mixture. Nonlimiting examples of buffers include PBS, Tris, MOPS (3-(N-morpholino)propanesulfonic acid) and Tricine. The hybridization may be conducted under the conditions to facilitate the binding of the single-stranded nucleic acid probe and the target nucleic acid, for example, at low temperatures and low salt concentrations. Those conditions to facilitate hybridization are known in the field. The target nucleic acid may be a single-stranded or double-stranded nucleic acid. For example, a double-stranded target nucleic acid may be denaturated into separate single strands, as described above. The target nucleic acid may be DNA or RNA.

The method of detecting Listeria monocytogenes includes contacting the hybridized product from the target nucleic acid fragment and the probe to a RNase H to cleave the probe, resulting in probe fragment dissociating from the hybridized product; and The hybridized product and the RNase H may contact each other in a liquid medium. A suitable liquid medium may be selected according to the requirement(s). The liquid medium may be, for example, water, a buffer, or a PCR mixture. Nonlimiting examples of buffers include PBS, Tris, MOPS (3-(N-morpholino)propanesulfonic acid) and Tricine. The contact may be conducted under substantially the same conditions as PCR conditions or in a PCR mixture.

The RNase H may be RNase HI or RNase HII. The RNase H may hydrolyze RNA in the RNA-DNA hybrid. For RNase H activity, a divalent ion (for example, Mg²⁺, Mn²⁺) is required. The RNase H cleaves RNA 3′-O—P linkages to produce 3′-hydroxyl and 5′-phosphate end products. The RNase H may be selected from the group consisting of a Pyrococcus furiosus RNase HII, a Pyrococcus horikoshi RNase HII, a Thermococcus litoralis RNase HI, and a Thermus thermophilus RNase HI. The Pyrococcus furiosus RNase HII may have an amino acid sequence of SEQ ID NO. 7. The RNase H may be thermostable. For example, the RNase H may retain its activity during a denaturation process in PCR. The RNase H may be a reversibly modified form of a thermostable RNase HII, which is inactive in its modified form and active in its unmodified form, wherein the modification is a coupling of the RNase HII to a ligand, crosslinking of the RNase HII, or chemical modification of the RNase HII, and wherein the enzymatic activity of the modified RNase HII is restored by heating or adjusting the pH of a sample containing the RNase HII.

Such dissociation may naturally occur due to the binding force of the strands that is weaken by the cleavage or may be facilitated by a factor, such as temperature elevation. For example, the PCR mixture may include an RNase H enzyme that will specifically cleave the RNA sequence portion of a RNA-DNA duplex. After cleavage, the two halves of the probe dissociate from a target amplicon at the reaction temperature and diffuse into the reaction buffer. As the donor and acceptors separate, FRET is reversed and donor emission can be monitored. Cleavage and dissociation regenerates a site for further probe binding. In this way it is possible for a single amplicon to serve as a target or multiple rounds of probe cleavage until the primer is extended through the probe binding site.

The method of detecting Listeria monocytogenes includes detecting the probe nucleic acid fragment. The detection of the probe nucleic acid fragment may be carried out by any of a variety of methods, which are appropriately chosen according to the detectable markers. Throughout the specification, the term “a detectable marker” and “a detectable label” are used interchangeably. For example, the size of reaction products may be analyzed to detect the labeled probe fragment. The analysis of the size of the probe nucleic acid fragment may be carried out by any known method, for example, gel electrophoresis, gradient sedimentation, size exclusion chromatography, or homochromatography. When the detectable label used is a FRET pair, the labeled probe fragment may be identified in-situ by spectroscopy, without performing size analysis. Thus, real-time detection of the labeled probe fragment is achievable.

The method of detecting Listeria monocytoenes may further include cultivating the sample containing Listeria monocytoenes in an enrichment medium before the amplification process, to enhance growth of the Listeria monocytoenes.

The enrichment medium used for the cultivation may have the following features. The enrichment medium may not contain one of esculin and peptone, or both. In another embodiment, the enrichment medium may contain esculin as long it does not interfere with any of the steps performed according to the embodiments of the invention, for example amplification of a target sequence or detecting the target sequence by cleaving the labeled probe and detecting the cleaved labeled probe. The enrichment medium may be a medium for enhancing growth of Listeria monocytoenes, containing, per 1 L of distilled water, about 10 to about 40 g of tryptic soy broth (TSB), about 1 to about 10 g of yeast extract (YE), and about 1 to about 15 g of lithium chloride. The enrichment medium may further contain at least one component selected from the group consisting of about 1 to about 10 g of beef extract (BE), or a vitamin mix containing about 0.01 to about 0.5 mg of riboflavin, about 0.5 to about 1.5 mg of thiamine and about 0.01 to about 1.5 mg of biotin; about 1 to about 5 g of pyruvate or a salt thereof; and about 0.01 to about 1 g of ferric ammonium citrate. The enriched medium may further contain a buffer compound. The buffer compound may include 3-(N-morpholino)propanesulfonic acid (MOPS) free acid and a sodium salt. For example, the enriched medium may contain about 4 g of MOPS free acid and about 7.1 g of sodium MOPS. Alternatively, the enriched medium may contain about 1 to about 10 mg of acriflavine, about 5 to about 15 mg of polymyxin B, about 10 to about 30 mg of ceftazidime, and about 10 to about 60 mg of nalidixic acid.

The enrichment medium may be a medium containing, per 1 L of distilled water, about 10 to about 40 g of tryptic soy broth (TSB), about 1 to about 10 g of yeast extract (YE), about 1 to about 10 g of lithium chloride; about 1 to about 10 g of beef extract (BE) and/or a vitamin mix containing about 0.01 to about 0.5 mg of riboflavin, about 0.5 to about 1.5 mg of thiamine, and about 0.01 to about 1.5 mg of biotin; about 1 to about 5 g of pyruvate or a salt thereof; about 0.01 to 1 g of ferric ammonium citrate; about 4 g of MOPS free acid and about 7.1 g of sodium MOPS; and about 1 to about 10 mg of acriflavine, about 5 to about 15 mg of polymyxin B, and about 10 to about 30 mg of ceftazidime. For example, the enrichment medium may be a medium containing 30 g of tryptic soy broth (TSB), 6 g of yeast extract, 1 g of esculin, 10 g of LiCl, 2 g of sodium pyruvate, 0.1 g of ferric ammonium citrate, 8 g of MOPS free acid, 14.2 g of MOPS, sodium, 5 g of beef extract, and a vitamin mix containing about 0.1 mg of riboflavin, about 1.0 mg of thiamine, and about 1.0 mg of biotin; or a medium (sometimes, referred to as A2.2 medium) containing, per 1 L of distilled water, about 30 g of tryptic soy broth (TSB), about 6 g of yeast extract (YE), about 1 to about 10 g of lithium chloride; 5 g of beef extract (BE) and/or a vitamin mix containing about 0.1 mg of riboflavin, about 1.0 mg of thiamine, and about 1.0 mg of biotin; 2 g of sodium pyruvate; about 0.1 g of ferric ammonium citrate; about 4 g of MOPS free acid and about 7.1 g of sodium MOPS; about 5 mg of acriflavine, about 10 mg of polymyxin B, and about 20 mg of ceftazidime. Using such an enrichment medium may eliminate or reduce PCR inhibitors in culture products and promote growth of Listeria species while inhibiting growth of background microflora, thus enabling efficient detection of Listeria monocytogenes in a sample.

Enrichment medium may be BHI (brain heart infusion) broth, which may be used as it is or supplemented with trace ingredients such as sodium chloride and/or disodium phosphate. BHI is commercially available from different sources, under different trade names such as BACTO®, BBL®, or Difco®. Enrichment medium may also be tryptic soy broth (TSB) with or without supplement of 0.6% yeast extract.

An exemplary protocol for detecting a target Listeria monocytogenes sequence may include the steps of providing a food sample or surface wipe, mixing the sample or wipe with a growth medium and incubating to increase the number or population of Listeria (“enrichment”), disintegrating Listeria cells (“lysis”), and subjecting the obtained lysate to amplification and detection of target Salmonella sequence. Food samples may include, but are not limited to, fish such as smoked salmon, dairy products such as milk and cheese, and liquid eggs, poultry, fruit juices, meats such as ground pork, pork, ground beef, or beef, or deli meat, vegetables such as spinach, or environmental surfaces such as stainless steel, rubber, plastic, and ceramic.

The limit of detection (LOD) for food contaminants is described in terms of the number of colony forming units (CFU) that can be detected in either 25 grams of solid or 25 mL of liquid food or on a surface of defined area. By definition, a colony-forming unit is a measure of viable bacterial numbers. Unlike indirect microscopic counts where all cells, dead and living, are counted, CFU measures viable cells. One CFU (one bacterial cell) will grow to form a single colony on an agar plate under permissive conditions. The United States Food Testing Inspection Service defines the minimum LOD as 1 CFU/25 grams of solid food or 25 mL of liquid food or 1 CFU/surface area.

In practice, it is impossible to reproducibly inoculate a food sample or surface with a single CFU and insure that the bacterium survives the enrichment process. This problem is overcome by inoculating the sample at either one or several target levels and analyzing the results using a statistical estimate of the contamination called the Most Probable Number (MPN). As an example, a Listeria culture can be grown to a specific cell density by measuring the absorbance in a spectrophotometer. Ten-fold serial dilutions of the target are plated on agar media and the numbers of viable bacteria are counted. This data is used to construct a standard curve that relates CFU/volume plated to cell density. For the MPN to be meaningful, test samples at several inoculum levels are analyzed. After enrichment and extraction a small volume of sample is removed for real-time analysis. The ultimate goal is to achieve a fractional recovery of between 25% and 75% (i.e. between 25% and 75% of the samples test positive in the assay using RT-PCR employing a CataCleave probe, which will be explained below). The reason for choosing these fractional recovery percentages is that they convert to MPN values of between 0.3 CFU and 1.375 CFU for 25 gram samples of solid food, 25 mL samples of liquid food, or a defined area for surfaces. These MPN values bracket the required LOD of 1 CFU/sample. With practice, it is possible to estimate the volume of diluted innoculum (based on the standard curve) to achieve these fractional recoveries.

Various embodiments will be described in further detail with reference to the following examples. These examples are for illustrative purposes only and are not intended to limit the scope of the invention.

### Example 1

### Real-Time PCR Amplification of L. Monocytogenes Using Primer Pair of SEQ ID NOs: 1 and 2 and Probe of SEQ ID NO: 3

A primer pair of SEQ ID NOs: 1 and 2 and a probe of SEQ ID NO: 3 were used to amplify and detect a target nucleic acid of Listeria monocytogenes in a sample according to real-time PCR amplification.

(1) Inclusivity Test

Sixty-one (61) strains of L. monocytogenes were cultivated overnight in a brain heart infusion medium at 35° C. 5 ul culture was lysed in 45 uL lysis solution. 2 ul lysate was added to a PCR mixture and subjected to real-time PCR. Real-time PCR was conducted in the presence of a primer pair of SEQ ID NOs: 1 and 2, and a probe of SEQ ID NO: 3. The PCR conditions and the PCR mixture composition were as follows.

PCR Conditions:

1 cycle of uNG incubation at 37° C. for 10 minutes;

1 cycle of initial denaturation at 95° C. for 10 minutes and 50 cycles of denaturation at 95° C. for 15 seconds; and

annealing/elongation at 60° C. for 20 seconds.

TABLE 1
Reaction mixture composition per well (μl)

| | Amount (μl) |
|---|---|
| 10× ICAN buffer | 2.5 |
| forward primer (20 μM) | 1 |
| reverse primer (20 μM) | 1 |
| CC probe (5 μM) | 1 |
| dN/UTP (2/4 mM, Fermentas) | 1 |
| Taq polymerase (5 U/μL) | 0.5 |
| UNG (10 U/μL) | 0.1 |
| Pfu RNase HII(5 U/μL) | 0.2 |
| Template DNA | 2 |
| Water | 15.70 |

In Table 1, ICAN buffer has the composition: 32 mM HELPES (pH 7.8), 100 mM potassium acetate, 4 mM magnesium acetate, 1% DMSO, 0.11% BSA. Forward primer and Reverse primer indicate primers of SEQ ID NOs: 1 and 2; and CC probe indicates a probe of SEQ ID NO: 3 with the 5′ end labeled with FAM and the 3′ end labeled with FAM® and BFQ (Black Hole Quencher), respectively.

The culture was subjected to lysis and the resulting lysate as a template was mixed into the PCR mixture. A total of 61 different L. monocytenges were used. A PCR mixture containing no template DNA was used as a negative control group. The list of L. monocytogenes strains used in the inclusivity test is shown in Table 2.

TABLE 2
Strains used in the inclusivity test.

| Name | serovar | Designations (Source) |
|---|---|---|
| Listeria monocytogenes | 1/2a | CDL 1 |
| Listeria monocytogenes | 1/2a | CDL 3 |
| Listeria monocytogenes | 1/2c | CDL 5 |
| Listeria monocytogenes | 1/2b | CDL 9 |
| Listeria monocytogenes | 3b | CDL 10 |
| Listeria monocytogenes | 4a | CDL 13 |
| Listeria monocytogenes | 4b | CDL 16 |
| Listeria monocytogenes | 4d/4e | CDL 18 |
| Listeria monocytogenes | 4d/4e | CDL 19 |
| Listeria monocytogenes | 4d/4e | CDL 21 |
| Listeria monocytogenes | 1/2b | CDL 35 |
| Listeria monocytogenes | 1/2c | CDL 36 |
| Listeria monocytogenes | 1/2c | CDL 37 |
| Listeria monocytogenes | 1/2c | CDL 38 |
| Listeria monocytogenes | 3a | CDL 39 |
| Listeria monocytogenes | 3a | CDL 41 |
| Listeria monocytogenes | 3b | CDL 42 |
| Listeria monocytogenes | 3b | CDL 43 |
| Listeria monocytogenes | 3b | CDL 45 |
| Listeria monocytogenes | 3c | CDL 47 |
| Listeria monocytogenes | 3c | CDL 48 |
| Listeria monocytogenes | 3c | CDL 49 |
| Listeria monocytogenes | 3c | CDL 50 |
| Listeria monocytogenes | 4a | CDL 51 |
| Listeria monocytogenes | 4a | CDL 111 |
| Listeria monocytogenes | 1/2b | CDL 112 |
| Listeria monocytogenes | 1/2c | CDL 113 |
| Listeria monocytogenes | 3b | CDL 114 |
| Listeria monocytogenes | 3b | CDL 115 |
| Listeria monocytogenes | 1/2a | CDL 116 |
| Listeria monocytogenes | 4a | CDL 117 |
| Listeria monocytogenes | 4b | CDL 118 |
| Listeria monocytogenes | 4d/e | CDL 120 |
| Listeria monocytogenes | 7 | CDL 122 |
| Listeria monocytogenes | 4b | CDL 123 |
| Listeria monocytogenes | 1/2b | CDL 125 |
| Listeria monocytogenes | 1/2b | CDL 128 |
| Listeria monocytogenes | 1/2b | CDL 131 |
| Listeria monocytogenes | 1/2b | CDL 132 |
| Listeria monocytogenes | 4b | CDL 136 |
| Listeria monocytogenes | 4b | CDL 137 |
| Listeria monocytogenes | 4b | CDL 138 |
| Listeria monocytogenes | 4b | CDL 139 |
| Listeria monocytogenes | 4b | CDL 140 |
| Listeria monocytogenes | 1/2b | CDL 142 |
| Listeria monocytogenes | 1/2b | CDL 143 |
| Listeria monocytogenes | 1/2a | CDL 144 |
| Listeria monocytogenes | 1/2a | CDL 145 |
| Listeria monocytogenes | 1/2b | CDL 147 |
| Listeria monocytogenes | 3b | CDL 149 |
| Listeria monocytogenes | 1/2c | ATCC 19112, CWD 106 |
| Listeria monocytogenes | 4c | ATCC 19116, CWD 108 |
| Listeria monocytogenes | 4b | CWD 1559 |
| Listeria monocytogenes | 3b | CWD 1591 |
| Listeria monocytogenes | 1/2b | CWD 1597 |
| Listeria monocytogenes | 3b | CWD 1600 |
| Listeria monocytogenes | 1/2a | CWD 1609 |
| Listeria monocytogenes | 4b | ATCC 51414, CWD 104 |
| Listeria monocytogenes | 4d | ATCC 19117, CWD 109 |
| Listeria monocytogenes | 4e | ATCC 19118, CWD 110 |
| Listeria monocytogenes | 1/2a | CWD 72 |

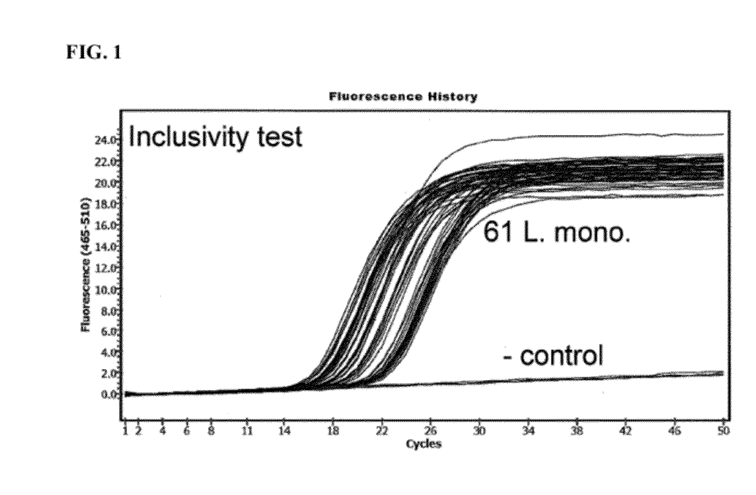

FIG. 1 is a graph illustrating the real-time PCR results on 61 L. monocytogenes strains. Referring to FIG. 1, all the 61 L. monocytogenes strains were successfully amplified in the real-time PCR using the primer pair of SEQ ID NOs: 1 and 2 and the probe of SEQ ID NO: 3. This result indicates that real-time PCR using the primer pair and the probe according to an embodiment is highly specific to L. monocytogenes strains.

(2) Exclusivity Test

Real-time PCR was carried out using the same PCR mixture composition under the same PCR conditions as in (1) Inclusivity Test, except that the 33 other Listeria species and 23 non-Listeria species were used as templates, instead of L. monocytogenes. The culture media were the same as those used in Inclusivity Test, but the temperature was adjusted to an optimum growth depending on individual strain.

33 other Listeria species used in the experiment include 5 species. The list of the test strains is shown below.

23 non-Listeria species used in the experiment include Bacillus mycoides, Brochothrix campestris, Carnobacterium divergens, Carnobacterium malaroma, Enterobacter aerogenes, Enterobacter cancerogenus, Enterobacter cloacae, Enterobacter intermedia, Enterobacter sakazkii, Escherichia coli, Escherichia coli O157:H7, Klebsiella pneumoniae, Kurthia zopfii, Lactococcus lactis, Proteus hauseri, Proteus mirabilis, Proteus vulgaris, Rhodococcus aqui, Staphylococcus aureus, Staphylococcus saprophyticus, Streptococcus agalactiae, Streptococcus dysgalactiae, and Streptococcus sanguinis. L. monocytogenes strains, L. innocua, L. welshimeri, L. seeligeri, L. ivanovii, and L. Gray were used as a positive control group. The 23 non-Listeria species and the positive control are listed in Table 3 and Table 4, respectively.

TABLE 3
List of non-Listeria species used in exclusivity test

| Organisms | Source | Origin | O₂ | Temp. ° C. |
|---|---|---|---|---|
| Bacillus mycoides | ATCC10206 | Unknown | | 30 |
| Brochothrix campestris | ATCC43754 | Soil | | 26 |
| Carnobacterium divergens | ATCC35677 | Beef | | 30 |
| Carnobacterium | ATCC43224 | Beef | | 26 |
| maltaromaticum | | | | |
| Enterobacter aerogenes | ATCC 13048 | Sputum | | 37 |
| Enterobacter cancerogenus | ATCC 35317 | Human | | 37 |
| Enterobacter cloacae | ATCC 13047 | Spinal | | 37 |
| | | Fluid | | |
| Enterobacter intermedia | ATCC 33110 | Water | | 37 |
| Enterobacter sakazakii | ATCC | Human | | 37 |
| | BAA-894 | | | |
| Escherichia coli | ATCC 11303 | | | 37 |
| Escherichia coli O157:H7 | ATCC 43895 | Meat | | 37 |
| Klebsiella pneumoniae | ATCC 13883 | | | 37 |
| Kurthia zopfii | ATCC33403 | Turkey | | 26 |
| Lactococcus lactis | ATCC 11454 | Unknown | | 37 |
| Proteus hauseri | ATCC13315 | | | 37 |
| Proteus mirabilis | ATCC35659 | | | 37 |
| Proteus vulgaris | ATCC33420 | Clinical | | 37 |
| | | Isolate | | |
| Rhodococcus equi | ATCC10146 | Horse | | 37 |
| Staphylococcus aureus | ATCC10832 | Unknown | | 37 |
| Staphylococcus epidermidis | ATCC 12228 | Unknown | | 37 |
| Staphylococcus saprophyticus | ATCC 15305 | Urine | | 37 |
| Streptococcus agalactiae | ATCC 12386 | Unknown | | 37 |
| Streptococcus dysgalactiae | ATCC 12388 | Human | | 37 |
| Streptococcus sanguinis | ATCC 10556 | Human | | 37 |

TABLE 4
Positive controls for exclusivity test

| Name | serovar | Designations |
|---|---|---|
| Listeria innocua | | L82/14 |
| Listeria innocua | | L82/16 |
| Listeria innocua | | L82/18 |
| Listeria innocua | 6a | DA-20, CWD 181 |
| Listeria welshimeri | 6a | CDL 209 |
| Listeria welshimeri | 6b | CDL 243 |
| Listeria welshimeri | | L82/2, LFD 5121 |
| Listeria welshimeri | | L82/10, LFD 5125 |
| Listeria welshimeri | | L21/44, LFD 782 |
| Listeria welshimeri | | L21/46, LFD 783 |
| Listeria welshimeri | | L21/40, LFD 860 |
| Listeria welshimeri | 6a | ATCC 35897, CWD 114 |
| Listeria seeligeri | 1/2b | CDL 84 |
| Listeria seeligeri | 4c | CDL 98 |
| Listeria seeligeri | 1/2b | ATCC 35967, CWD 166 |
| Listeria ivanovii | 5 | L45/74, LFD 2949 |
| Listeria ivanovii | | L24/6 |
| Listeria ivanovii | | L24/22, LFD 891 |
| Listeria ivanovii | 5 | ATCC 19119, CWD 164 |
| Listeria grayi | | ATCC 25400, CWD 671 |
| Listeria grayi | | ATCC 25401, CWD 673 |
| Listeria grayi | | ATCC 25402, CWD 20 |
| Listeria grayi | | ATCC 25403, CWD 672 |
| Listeria grayi | | ATCC 19120, CWD 2091 |

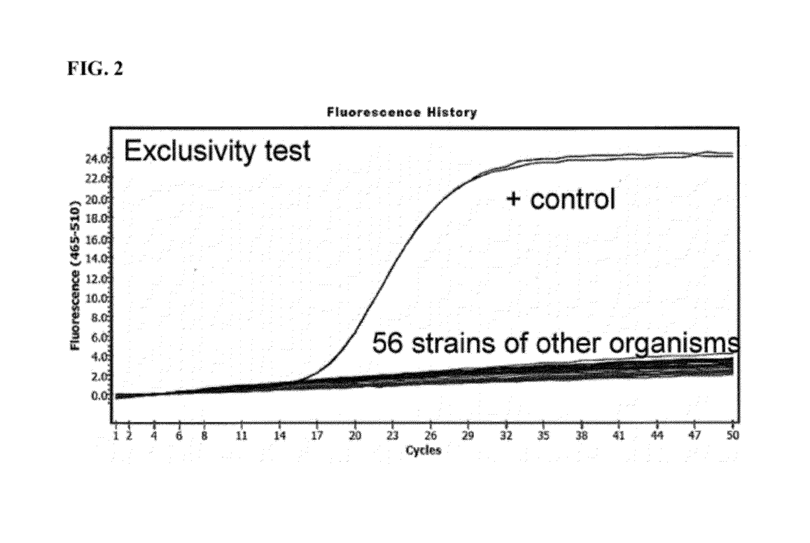

FIG. 2 is a graph illustrating the real-time PCR results on 56 other organisms. Referring to FIG. 2, none of the 23 non-Listeria species and 33 other Listeria species were amplified in the real-time PCR using the primer pair of SEQ ID NOs: 1 and 2 and the probe of SEQ ID NO: 3. However, the positive control group was amplified. These results indicate that real-time PCR using the primer pair and probe according to an embodiment is highly specific to L. monocytenges strains.

(3) Detection Limit Test

Real-time PCR was carried out by adding the plasmid containing target sequences of the full-length L. monocytogenes inlA gene as template DNA, which is prepared by serially diluting the plasmid solution, so as to be 10⁶ to 10⁰ copies per μL of plasmid to determine the detection limit. The PCR condition and the PCR mixture composition were the same as in (1) Inclusivity Test, except that the concentration of plasmid was varied.

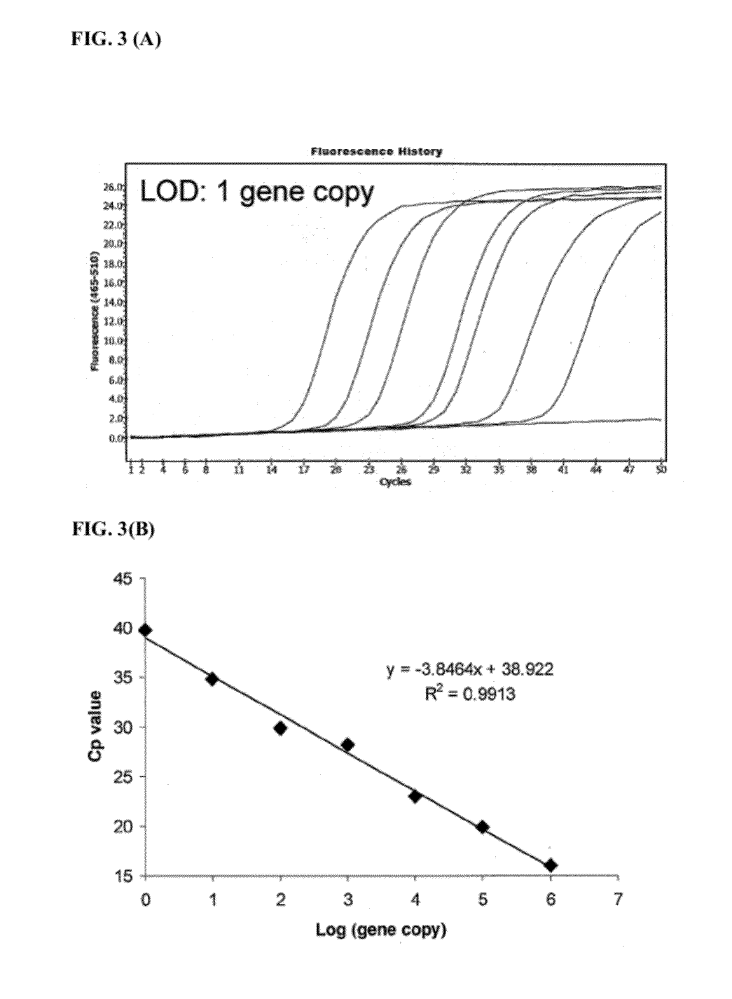

FIGS. 3(A) and 3(B) are graphs illustrating the real-time PCR amplification results with respect to the number of copies of plasmid containing the target sequences of the L. monocytogenes gene and Listeria genus. FIG. 3(A) shows fluorescence and FIG. 3(B) shows Cp value. As illustrated in FIGS. 3(A) and 3(B), up to one copy of plasmid per reaction could be detected.

Also, the real-time PCR was carried out by adding the plasmid containing the L. monocytogenes strains, which is prepared by serially diluting the plasmid solution so as to be 10⁹ to 10³ cfu per ml of plasmid to determine the detection limit. The PCR condition and the PCR mixture composition were the same as in the above-described Inclusivity Test, except that the concentration of plasmid was varied. The template DNA in the PCR mixture was lysate obtained by lysis of L. monocytogenes culture using a TZ buffer.

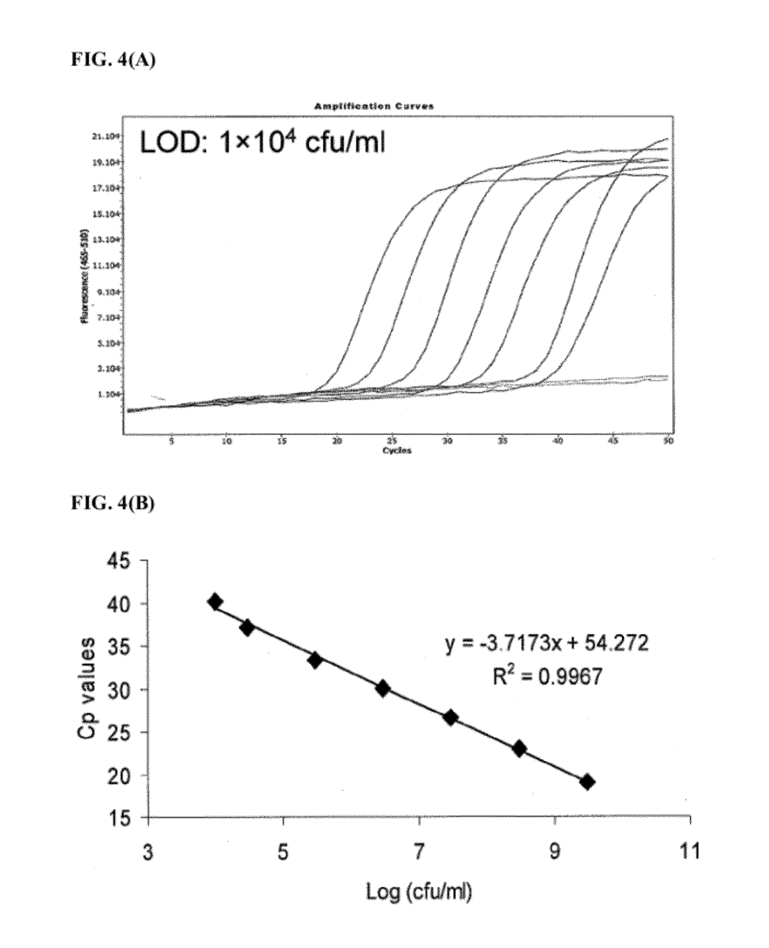

FIGS. 4(A) and 4(B) are graphs illustrating the real-time PCR amplification results with respect to the number of strains of L. monocytogenes. As illustrated in FIGS. 4(A) and 4(B), up to 10⁴ cfu/ml strains of L. monocytogenes could be detected.

The results of FIGS. 3(A) through 4(B) indicate that the real-time PCR amplification and detection of target sequences of the L. monocytogenes using the primer pair and probe according to an embodiment is suitable to detect L. monocytogenes gene in a sample at a high sensitivity.

### Example 2

### Specific Detection of L. Monocytogenes in Contaminated Sample

A sample contaminated with L. monocytogenes was subjected to real-time PCR to amplify a target nucleic acid of L. monocytogenes in the presence of a primer pair of SEQ ID NOs: 1 and 2 and a probe of SEQ ID NO: 3 to detect L. monocytogenes in the sample.

Milk, cheese, deli meat (ham), liquid egg, frankfurters, ground beef, spinach, smoked salmon, strainless steel, rubber, plastic, or ceramic tile was used as the contaminated sample.

(1) Detection of L. Monocytogenes in Contaminated Ceramic Tile

L. monocytogenes 4b was diluted with 0.5% non-fat milk to concentrations of 1 cfu and 10 cfu. 1 mL of each dilution was inoculated on a 1 inch×1 inch ceramic tile surface and air-dried overnight at room temperature. The contaminant on each sample surface was collected with a phosphate buffered saline (PBS)-soaked sponge and then cultivated in 10 mL of an enrichment medium A2.2 (30 g/L of TSB, 6 g/L of yeast extract, 10 g/L of LiCl 10, 2 g/L of sodium pyruvate, 0.1 g/L of ferric ammonium citrate, 8 g/L of MOPS free acid, 14.2 g/L of MOPS sodium, 5 g/L of beef extract, a vitamin mix containing about 0.1 mg/L of riboflavin, about 1.0 mg/L of thiamine, and about 1.0 mg/L of biotin), which may optionally contain 1 g/L of esculin, or an enriched UVM-1 medium (5 g/L of Proteose peptone, 5 g/L of Tryptone/casein dig., 5 g/l of BE, 5 g/L of YE, 20 g/L of NaCl, 12 g/L of Na₂HPO₄ 2H₂O, 1.35 g/L of KH₂PO₄, 1 g/L of esculin, 0.012 g/L of acroflavine, and 0.02 g/L of nalidixic acid) at 35° C. for 24 hours.

L. monocytogenes cultures grown in the medium A2.2 and the UVM-1 medium, respectively, were subjected to real-time PCR amplification in the presence of a primer pair of SEQ ID NOs: 1 and 2 and a probe of SEQ ID NO: 3. The PCR results are shown in Table 5 below.

TABLE 5
Detection of L.monocytogenes in contaminated ceramic tile (Cp value)

| | Medium A2.2 | | Medium UVM-1 | |
|---|---|---|---|---|
| Sample | 1 cfu | 10 cfu | 1 cfu | 10 cfu |
| 1 | 17.62 | 18.68 | — | 31.03 |
| 2 | 17.16 | 20.09 | 35.22 | 35.94 |
| 3 | 19.83 | 17.06 | 38.94 | 31.08 |
| 4 | 17.85 | 17.28 | — | 36.97 |
| 5 | 16.97 | 16.91 | 35.5 | 35.37 |
| 6 | — | 17.38 | 32.73 | 34.51 |
| 7 | — | 19.63 | — | 39.87 |
| 8 | 17.78 | 21.27 | 38.12 | 40.84 |
| Average | 17.9 | 18.5 | 36.1 | 35.37 |
| Positive ratio | 0.75 | 1 | 0.625 | 1 |

Referring to Table 5, in the enriched medium A2.2, positive ratios were 0.75 and 1 in 1 cfu and 10 cfu, respectively. In the enriched UVM-1 medium, positive ratio were 0.625 and 1 in 1 cfu and 10 cfu, respectively. Also, in the enriched medium A2.2, average Cp values are 17.9 (1 cfu) and 18.5 (10 cfu), whereas in the enriched UVM-1 medium, average Cp values are 36.1 (1 cfu) and 35.37 (10 cfu). The results indicate that L. monocytogenes could be detected at a high sensitivity in the enriched medium A2.2 compared with the enriched UVM-1 medium.

(2) Detection of L. Monocytogenes in Contaminated Rubber

L. monocytogenes was diluted with 0.5% non-fat milk to concentrations of 3.3 cfu and 33 cfu. 1 mL of each dilution was inoculated on a 1 inch×1 inch rubber surface and air-dried overnight at room temperature. Other conditions in this experiment were the same as in (1) above. The PCR results are shown in Table 6 below.

TABLE 6
Detection of L. monocytogenes in contaminated rubber (Cp value)

| | Medium A2.2 | | Medium UVM-1 | |
|---|---|---|---|---|
| Sample | 3.3 cfu | 33 cfu | 3.3 cfu | 33 cfu |
| 1 | — | — | — | — |
| 2 | 40.94 | — | — | — |
| 3 | 40.46 | — | — | — |
| 4 | — | 40.47 | — | — |
| 5 | 40.81 | 39.79 | — | 40.51 |
| 6 | — | — | — | — |
| 7 | — | — | — | — |
| 8 | — | — | — | — |
| Average | 40.7 | 40.1 | — | 40.5 |
| Positive ratio | 0.375 | 0.25 | 0 | 0.125 |

(3) Detection of L. Monocytogenes in Contaminated Milk

Milk was inoculated with L. monocytogenes 1/2a to concentrations of about 1 cfu/25 g and about 5 cfu/25 g. After incubation of the samples at 4° C. for 24 hours, 10 ml of each sample was cultivated in an enriched medium A2.2 medium at 30° C. for 24 hours. Other conditions in this experiment were the same as in (1) above. According to the PCR results, the average Cp values were 36.8 and 33.6, respectively and positive ratios were 0.5 and 1, respectively.

(4) Detection of L. Monocytogenes in Contaminated Ham

25 g of smoked ham was inoculated with L. monocytogenes 4c to concentrations of about 1 cfu/25 and about 5 cfu/25 g. After incubation of the samples at 4° C. for 24 hours, 25 g of each sample was cultivated in an enriched medium A2.2 medium at 30° C. for 24 hours. Each MPN tube (0.1 g, 1 g, and 10 g) was cultivated in an enriched UVM-1 medium at 30° C. for 24 hours. Other conditions in this experiment were the same as in (1) above. According to the PCR results, the average Cp values were 30.3 and 26.4, respectively and positive ratios were 0.875 and 1, respectively. MPN(/25 g) was 0.75 cfu/25 g.

(5) Detection of L. Monocytogenes in Contaminated Egg

Liquid egg was inoculated with L. monocytogenes 1/2b to concentrations of about 1 cfu/25 ml and about 3 cfu/25 ml. After incubation of the samples at 4° C. for 24 hours, 251 of each sample was cultivated in an enriched medium A2.2 medium at 30° C. for 24 hours. Also, other samples used to determine MPN were cultivated in an enriched UVM-1 medium at 30° C. for 24 hours. Other conditions in this experiment were the same as in (1) above. According to the PCR results, the average Cp value was 33.2 and positive ratio was 0.125.

Any patent, patent application, publication, or other disclosure material identified in the specification is hereby incorporated by reference herein in its entirety. Any material, or portion thereof, that is said to be incorporated by reference herein, but which conflicts with existing definitions, statements, or other disclosure material set forth herein is only incorporated to the extent that no conflict arises between that incorporated material and the present disclosure material.

What is claimed is:

1. A composition comprising:
a first oligonucleotide of the sequence of SEQ ID NO: 1, and
a second oligonucleotide of the sequence of SEQ ID NO: 2.

2. The composition according to claim 1, further comprising a third oligonucleotide comprising a DNA sequence and an RNA sequence, said third oligonucleotide being the sequence of SEQ ID NO: 6 or SEQ ID NO: 8:
TGAGACCGTGTCTrGTTACATTCG (SEQ ID NO: 6), wherein the nucleotide “rG” at position 14 is a ribonucleotide, and
CGAATGTAACAGACACGGTCTCA (SEQ ID NO: 8), wherein at least one of the ribonucleotides at positions 9, 10, 11, 12, and 13 is a ribonucleotide.

3. The composition according to claim 2, wherein the third oligonucleotide is one or more selected from the group consisting of oligonucleotides of SEQ ID NOs: 3-6:
CGAATGTAArCAGACACGGTCTCA (SEQ ID NO: 3), wherein “rC” at position 10 is a ribonucleotide,
CGAATGTArArCrArGACACGGTCTCA (SEQ ID NO: 4), wherein “rA,” “rC,” “rA,” and “rG” at positions 9, 10, 11, and 12, respectively, are ribonucleotides,
CGAATGTAArCrArGrACACGGTCTCA (SEQ ID NO: 5), wherein “rC,” “rA,” “rG,” and “rA” at positions 10, 11, 12, and 13, respectively, are ribonucleotides, and
TGAGACCGTGTCTrGTTACATTCG (SEQ ID NO: 6), wherein the nucleotide “rG” at position 14 is a ribonucleotide.

4. The composition according to claim 2, wherein the third oligonucleotide is labeled with a detectable marker.

5. The composition according to claim 4, wherein the third oligonucleotide is labeled with a fluorescence resonance energy transfer (FRET) pair.

6. The composition according to claim 3, comprising the first oligonucleotide of SEQ ID NO. 1, the second oligonucleotide of SEQ ID NO. 2, and the third oligonucleotide of SEQ ID NO. 3.

7. A kit for detecting Listeria monocytogenes in a sample, the kit comprising
(a) a first primer of the sequence of SEQ ID NO: 1:
(b) a second primer of the sequence of SEQ ID NO: 2:
(c) a probe comprising an RNA sequence and a DNA sequence that are substantially complimentary to a target Listeria motocytogenes gene, and coupled to a detectable label.

8. The kit according to claim 7, wherein the target Listeria motocytogenes gene is internalin A gene or an RNA complementar to the internalin A.

9. The kit according to claim 7, further comprising
(d) an amplifying activity for a PCR amplification of the target DNA sequence to produce a Listeria monocytogenes PCR fragment; and
(e) an RNase H activity.

10. The kit according to claim 9, further comprising positive, internal, and negative controls.

11. The kit according to claim 10, further comprising uracil-N-glycosylase.

12. The kit according to claim 7, wherein the probe is coupled to a detectable label at both of its 3′-end and 5′-end.

13. The kit according to claim 12, wherein the detectable label is a fluorescent label.

14. The kit according to claim 13, wherein the probe is labeled with a FRET pair.

15. The kit according to claim 7, wherein the probe is linked to a solid support.

16. The kit according to claim 7, wherein the probe is in free form in a solution.

17. The kit according to claim 7, which further comprises an amplification buffer.

18. The kit according to claim 7, which further comprises an amplifying polymerase activity.

19. The kit according to claim 9, wherein the RNase H activity is the activity of a thermostable RNase H.

20. The kit according to claim 9, wherein the RNase H activity is a hot start RNase H activity.

21. The kit according to claim 7, wherein the probe comprises the sequence of SEQ ID NO: 6 or SEQ ID NO: 8:
TGAGACCGTGTCTrGTTACATTCG (SEQ ID NO: 6), wherein the nucleotide “rG” at position 14 is a ribonucleotide, and
CGAATGTAACAGACACGGTCTCA (SEQ ID NO: 8), wherein at least one of the ribonucleotides at positions 9, 10, 11, 12, and 13 is a ribonucleotide.

22. The kit according to claim 21, wherein the probe is one or more selected from the group consisting of oligonucleotides of SEQ ID NOs: 3-6:
CGAATGTAArCAGACACGGTCTCA (SEQ ID NO: 3), wherein “rC” at position 10 is a ribonucleotide,
CGAATGTArArCrArGACACGGTCTCA (SEQ ID NO: 4), wherein “rA,” “rC,” “rA,” and “rG” at positions 9, 10, 11, and 12, respectively, are ribonucleotides,
CGAATGTAArCrArGrACACGGTCTCA (SEQ ID NO: 5), wherein “rC,” “rA,” “rG,” and “rA” at positions 10, 11, 12, and 13, respectively, are ribonucleotides, and
TGAGACCGTGTCTrGTTACATTCG (SEQ ID NO: 6), wherein the nucleotide “rG” at position 14 is a ribonucleotide.

23. A method of detecting Listeria monocytogenes in a sample, the method comprising:
(a) amplifying a target nucleic acid of Listeria monocytogenes in the sample to produce an increased number of copies of the target nucleic acid, the amplifying including hybridizing a first primer of SEQ ID NO: 1 and a second primer of SEQ ID NO: 2 to the target nucleic acid in the sample to obtain a hybridized product of the target nucleic acid and the primers, and extending the first and the second primers of the hybridized product using a template-dependent nucleic acid polymerase to produce an extended primer product;
(b) hybridizing the target nucleic acid to at least one probe oligonucleotide which is capable of being hybridized to the target nucleic acid to obtain a hybridized product of the target nucleic acid:probe oligonucleotide, wherein the probe comprises a DNA sequence and an RNA sequence and is coupled to a detectable label;
(c) contacting the hybridized product of the target nucleic acid:the probe oligonucleotide to an RNase H to cleave the probes; and
(d) detecting an increase in the emission of a signal from the label on the probe, wherein the increase in signal indicates the presence of the Listeria monocytogenes target nucleic acid in the sample.

24. The method according to claim 23, wherein the probe oligonucleotide is the oligonucleotide of SEQ ID NO: 3 or 6:
TGAGACCGTGTCTrGTTACATTCG (SEQ ID NO: 6), wherein the nucleotide “rG” at position 14 is a ribonucleotide, and
CGAATGTAACAGACACGGTCTCA (SEQ ID NO: 8), wherein at least one of the ribonucleotides at positions 9, 10, 11, 12, and 13 is a ribonucleotide.

25. The method according to claim 24, wherein the probe oligonucleotide is selected from the group consisting of oligonucleotides of SEQ ID NOs: 3-6:
CGAATGTAArCAGACACGGTCTCA (SEQ ID NO: 3), wherein “rC” at position 10 is a ribonucleotide,
CGAATGTArArCrArGACACGGTCTCA (SEQ ID NO: 4), wherein “rA,” “rC,” “rA,” and “rG” at positions 9, 10, 11, and 12, respectively, are ribonucleotides,
CGAATGTAArCrArGrACACGGTCTCA (SEQ ID NO: 5), wherein “rC,” “rA,” “rG,” and “rA” at positions 10, 11, 12, and 13, respectively, are ribonucleotides, and
TGAGACCGTGTCTrGTTACATTCG (SEQ ID NO: 6), wherein the nucleotide “rG” at position 14 is a ribonucleotide.

26. The method according to claim 23, wherein the detectable label is a fluorescence resonance energy transfer pair.

27. The method according to claim 23, wherein the amplifying is conducted using a method selected from the group consisting of Polymerase Chain Reaction, Ligase Chain Reaction, Self-Sustained Sequence Replication, Strand Displacement Amplification, Transcriptional Amplification System, Q-Beta Replicase, Nucleic Acid Sequence Based Amplification, Cleavage Fragment Length Polymorphism, Isothermal and Chimeric Primer-initiated Amplification of Nucleic Acid, and Ramification-extension Amplification Method.

28. The method according to claim 23, wherein the amplifying, the hybridizing and the contacting are simultaneously or sequentially carried out.

29. The method according to claim 23, further comprising cultivating the sample containing Listeria monocytogenes in an enrichment medium before the amplifying, to enhance growth of the Listeria monocytogenes.

30. The method according to claim 29, wherein the enrichment medium containing, per 1 L of distilled water, about 10 to about 40 g of tryptic soy broth, about 1 to about 10 g of yeast extract, and about 1 to about 10 g of lithium chloride.

31. The method according to claim 30, wherein the enrichment medium further comprises at least one component selected from the group consisting of about 1 to about 10 g of beef extract, and/or a vitamin mix containing about 0.01 to about 0.5 mg of riboflavin, about 0.5 to about 1.5 mg of thiamine and about 0.01 to about 1.5 mg of biotin; about 1 to about 5 g of pyruvate or a salt thereof; and about 0.01 to about 1 g of ferric ammonium citrate.

32. The method according to claim 31, wherein the enrichment medium further comprises a buffer compound.

33. The method according to claim 32, wherein the buffer compound comprises 3-(N-morpholino)propanesulfonic acid (MOPS) and a sodium salt thereof.

34. The method according to claim 33, wherein the buffer compound comprises about 4 g of MOPS and about 7.1 g of sodium MOPS.

35. The method according to claim 29, wherein the enrichment medium comprises about 1 to about 10 mg of acriflavine, about 5 to about 15 mg of polymyxin B, and about 10 to about 30 mg of ceftazidime.

36. The method according to claim 29, wherein the enrichment medium comprises, per 1 L of distilled water, about 10 to about 40 g of tryptic soy broth, about 1 to about 10 g of yeast extract, about 1 to about 10 g of lithium chloride; about 1 to about 10 g of beef extract and/or a vitamin mix containing about 0.01 to about 0.5 mg of riboflavin, about 0.5 to about 1.5 mg of thiamine, and about 0.01 to about 1.5 mg of biotin; about 1 to about 5 g of pyruvate or a salt thereof; about 0.1 to about 1 g of ferric ammonium citrate; about 4 g of 3-(N-morpholino)propanesulfonic acid (MOPS) and about 7.1 g of sodium MOPS; and about 1 to about 10 mg of acriflavine, about 5 to about 15 mg of polymyxin B, and about 10 to about 30 mg of ceftazidime.

37. The method according to claim 29, wherein the enrichment medium does not comprise one of esculin or peptone, or both.

38. The method according to claim 29, wherein the enrichment medium comprises, per 1 L of distilled water, about 30 g of tryptic soy broth, about 6 g of yeast extract, about 1 to about 10 g of lithium chloride; about 5 g of beef extract and/or a vitamin mix containing about 0.1 mg of riboflavin, about 1.0 mg of thiamine, and about 1.0 mg of biotin; about 2 g of sodium pyruvate; about 0.2 g of ferric ammonium citrate; about 4 g of 3-(N-morpholino)propanesulfonic acid (MOPS) and about 7.1 g of sodium MOPS; and about 5 mg of acriflavine, about 10 mg of polymyxin B, and about 20 mg of ceftazidime.

39. The method according to claim 29, wherein the enrichment medium is a tryptic soy broth supplemented with a yeast extract, or a brain-heart infusion broth.

40. The method according to claim 23, wherein the sample is food or a surface wipe.

41. A method of detecting Listeria monocytogenes in a sample, the method comprising:
(a) reverse transcribing the Listeria monocytogenes target RNA in the presence of a reverse transcriptase activity and the reverse amplification primer to produce a target cDNA of the target RNA;
(b) amplifying the target cDNA sequence to produce an increased number of copies of the target nucleic acid, the amplifying including hybridizing a first primer of SEQ ID NO: 1 and a second primer of SEQ ID NO: 2 to the target cDNA to obtain a hybridized product of the target nucleic acid and the primers, and extending the first and the second primers of the hybridized product using a template-dependent nucleic acid polymerase to produce an extended primer product;
(c) hybridizing the target nucleic acid to at least one probe oligonucleotide which is substantially complimentary to the target cDNA to obtain a hybridized product of the target nucleic acid:probe oligonucleotide, wherein the probe comprises a DNA sequence and an RNA sequence and is coupled to a detectable label;
(d) contacting the hybridized product of the target nucleic acid:probe oligonucleotide to an RNase H to cleave the probes; and
(e) detecting an increase in the emission of a signal from the label on the probe, wherein the increase in signal indicates the presence of the Listeria monocytogenes target RNA in the sample.

42. The method according to claim 41, wherein the probe oligonucleotide is the oligonucleotide of SEQ ID NO: 6 or 8:
TGAGACCGTGTCTrGTTACATTCG (SEQ ID NO: 6), wherein the nucleotide “rG” at position 14 is a ribonucleotide, and
CGAATGTAACAGACACGGTCTCA (SEQ ID NO: 8), wherein at least one of the ribonucleotides at positions 9, 10, 11, 12, and 13 is a ribonucleotide.

43. The method according to claim 42, wherein the probe oligonucleotide is selected from the group consisting of oligonucleotides of SEQ ID NOs: 3-6:
CGAATGTAArCAGACACGGTCTCA (SEQ ID NO: 3), wherein “rC” at position 10 is a ribonucleotide,
CGAATGTArArCrArGACACGGTCTCA (SEQ ID NO: 4), wherein “rA,” “rC,” “rA,” and “rG” at positions 9, 10, 11, and 12, respectively, are ribonucleotides,
CGAATGTAArCrArGrACACGGTCTCA (SEQ ID NO: 5), wherein “rC,” “rA,” “rG,” and “rA” at positions 10, 11, 12, and 13, respectively, are ribonucleotides, and
TGAGACCGTGTCTrGTTACATTCG (SEQ ID NO: 6), wherein the nucleotide “rG” at position 14 is a ribonucleotide.

44. The method according to claim 41, wherein the detectable marker is a fluorescence resonance energy transfer pair.

45. The method according to claim 41, wherein the amplifying is conducted using a method selected from the group consisting of Polymerase Chain Reaction, Ligase Chain Reaction, Self-Sustained Sequence Replication, Strand Displacement Amplification, Transcriptional Amplification System, Q-Beta Replicase, Nucleic Acid Sequence Based Amplification, Cleavage Fragment Length Polymorphism, Isothermal and Chimeric Primer-initiated Amplification of Nucleic Acid, and Ramification-extension Amplification Method.

46. The method according to claim 41, wherein the amplifying, the hybridizing and the contacting are simultaneously or sequentially carried out.

47. The method according to claim 41, further comprising cultivating the sample containing Listeria monocytogenes in an enrichment medium before the amplifying, to enhance growth of the Listeria monocytogenes

48. The method according to claim 47, wherein the enrichment medium contains, per 1 L of distilled water, about 10 to about 40 g of tryptic soy broth, about 1 to about 10 g of yeast extract, and about 1 to about 10 g of lithium chloride.

49. The method according to claim 48, wherein the enrichment medium further comprises at least one component selected from the group consisting of about 1 to about 10 g of beef extract, and/or a vitamin mix containing about 0.01 to about 0.5 m g of riboflavin, about 0.5 to about 1.5 mg of thiamine and about 0.01 to about 1.5 mg of biotin; about 1 to about 5 g of pyruvate or a salt thereof; and about 0.01 to about 1 g of ferric ammonium citrate.

50. The method according to claim 49, wherein the enrichment medium further comprises a buffer compound.

51. The method according to claim 50, wherein the buffer compound comprises 3-(N-morpholino)propanesulfonic acid (MOPS) and a sodium salt thereof.

52. The method according to claim 50, wherein the buffer compound comprises about 4 g of MOPS and about 7.1 g of sodium MOPS.

53. The method according to claim 47, wherein the enrichment medium comprises about 1 to about 10 mg of acriflavine, about 5 to about 15 mg of polymyxin B, and about 10 to about 30 mg of ceftazidime.

54. The method according to claim 47, wherein the enriched medium comprises, per 1 L of distilled water, about 10 to about 40 g of tryptic soy broth, about 1 to about 10 g of yeast extract, about 1 to about 10 g of lithium chloride; about 1 to about 10 g of beef extract and/or a vitamin mix containing about 0.01 to about 0.5 mg of riboflavin, about 0.5 to about 1.5 mg of thiamine, and about 0.01 to about 1.5 mg of biotin; about 1 to about 5 g of pyruvate or a salt thereof; about 0.1 to about 1 g of ferric ammonium citrate; about 4 g of 3-(N-morpholino)propanesulfonic acid (MOPS) and about 7.1 g of sodium MOPS; and about 1 to about 10 mg of acriflavine, about 5 to about 15 mg of polymyxin B, and about 10 to about 30 mg of ceftazidime.

55. The method according to claim 47, wherein the enrichment medium does not comprise one of esculin or peptone, or both.

56. The method according to claim 47, wherein the enriched medium comprises, per 1 L of distilled water, about 30 g of tryptic soy broth, about 6 g of yeast extract, about 1 to about 10 g of lithium chloride; about 5 g of beef extract and/or a vitamin mix containing about 0.1 mg of riboflavin, about 1.0 mg of thiamine, and about 1.0 mg of biotin; about 2 g of sodium pyruvate; about 0.2 g of ferric ammonium citrate; about 4 g of 3-(N-morpholino)propanesulfonic acid (MOPS) and about 7.1 g of sodium MOPS; and about 5 mg of acriflavine, about 10 mg of polymyxin B, and about 20 mg of ceftazidime.

57. The method according to claim 47, wherein the enrichment medium is a tryptic soy broth supplemented with a yeast extract, or a brain-heart infusion broth.

58. The method according to claim 41, wherein the sample is food or a surface wipe.

59. The kit according to claim 7, further comprising
(d) a reverse transcriptase activity for reverse transcription of the target Listeria monocytogenes,
(e) an amplifying activity for a PCR amplification of the target DNA sequence to produce a Listeria monocytogenes PCR fragment; and
(f) an RNase H activity.

60. The kit according to claim 59, further comprising positive, internal, and negative controls.

61. The kit according to claim 60, further comprising uracil-N-glycosylase.

62. The kit according to claim 59, wherein the probe is coupled to a detectable label at both of its 3′-end and 5′-end.

63. The kit according to claim 62, wherein the detectable label is a fluorescent label.

64. The kit according to claim 63, wherein the probe is labeled with a FRET pair.

65. The kit according to claim 59, wherein the probe is linked to a solid support.

66. The kit according to claim 59, wherein the probe is in free form in a solution.

67. The kit according to claim 59, which further comprises an amplification buffer.

68. The kit according to claim 59, which further comprises an amplifying polymerase activity.

69. The kit according to claim 59, wherein the RNase H activity is the activity of a thermostable RNase H.

70. The kit according to claim 59, wherein the RNase H activity is a hot start RNase H activity.

71. The kit according to claim 59, wherein the probe oligonucleotide is the oligonucleotide of SEQ ID NO: 3 or 6:
TGAGACCGTGTCTrGTTACATTCG (SEQ ID NO: 6), wherein the nucleotide “rG” at position 14 is a ribonucleotide, and
CGAATGTAACAGACACGGTCTCA (SEQ ID NO: 8), wherein at least one of the ribonucleotides at positions 9, 10, 11, 12, and 13 is a ribonucleotide.

72. The method according to claim 59, wherein the probe oligonucleotide is selected from the group consisting of oligonucleotides of SEQ ID NOs: 3-6:
CGAATGTAArCAGACACGGTCTCA (SEQ ID NO: 3), wherein “rC” at position 10 is a ribonucleotide,
CGAATGTArArCrArGACACGGTCTCA (SEQ ID NO: 4), wherein “rA,” “rC,” “rA,” and “rG” at positions 9, 10, 11, and 12, respectively, are ribonucleotides,
CGAATGTAArCrArGrACACGGTCTCA (SEQ ID NO: 5), wherein “rC,” “rA,” “rG,” and “rA” at positions 10, 11, 12, and 13, respectively, are ribonucleotides, and
TGAGACCGTGTCTrGTTACATTCG (SEQ ID NO: 6), wherein the nucleotide “rG” at position 14 is a ribonucleotide.
